(19) United States
(12) Patent Application Publication
(10) Pub. No.: US 2011/0009705 A1
(43) Pub. Date: Jan. 13, 2011

(54) INTERNAL TISSUE RETRACTION DEVICE, METHOD OF USE, AND SYSTEM

(75) Inventors: David L. Bombard, Edwards, CO (US); Brian White, Denver, CO (US)

Correspondence Address: Neugeboren O'Dowd PC, 1227 Spruce Street, SUITE 200, BOULDER, CO, 80302, US

(73) Assignee: HIPCO, LLC, Edwards, CO (US)

(21) Appl. No.: 12/652,113

(22) Filed: Jan. 5, 2010

Related U.S. Application Data
(60) Provisional application No. 61/142,757, filed on Jan. 6, 2009.

(51) Int. Cl. A61B 1/32

(52) U.S. Cl. 600/210

(57) ABSTRACT

A device for retracting internal tissue comprises a base section and an elongated section adapted to fit within a surgical portal coupled to and extending away from the base section. The elongated section comprises an elongated section tip, a top section, an outer bottom shell, an inner cavity, and an extendable gripping mechanism. The extendable gripping mechanism is adapted to hold and release internal tissue and comprises a telescoping section and a biasing device. The biasing device in one embodiment is substantially located in the cavity and comprises a proximal end operatively coupled to at least one of the base section, top section, and the outer shell. The biasing device also comprises a distal end operatively coupled to the telescoping section.

### PRIORITY AND CROSS REFERENCE TO RELATED APPLICATIONS

The present application claims the benefit of Provisional U.S. Patent Application No. 61/142,757 filed Jan. 6, 2009. The details of Application No. 61/142,757 are incorporated by reference into the present application in their entirety and for all proper purposes.

### FIELD OF THE INVENTION

Aspects of the present invention relate generally to medical devices. In particular, but not by way of limitation, aspects of the present invention relate to internal tissue retraction devices, methods of use, and systems.

### BACKGROUND OF THE INVENTION

During invasive medical procedures, internal tissue often disrupts the ability to quickly and smoothly complete the procedure. For example, during hip arthroscopy procedures, a capsulotomy may be created. The resultant free edges of the hip joint capsule tissue can obstruct the field of view of the arthroscope and impede the use of arthroscopic instruments. Other endoscopic and non-endoscopic procedures beside hip arthroscopy procedures may also be disrupted by internal tissue.

In order to appropriately complete an invasive surgical procedure such as hip arthroscopy, diminishing the internal tissue obstruction and allowing the appropriate instruments access to the procedure area is necessary. Current devices, methods, and systems do not adequately deal with the problems associated with internal tissue obstructions. For example, current devices, systems, and methods do not allow for the secured retraction of internal tissue with a device that may be left in place during the procedure without requiring continued adjustment of the device during the procedure. Moreover, current devices, systems, and methods do not allow for the easy use of surgical instruments after removing the internal tissue obstruction.

### SUMMARY OF THE INVENTION

Exemplary embodiments of the present invention that are shown in the drawings are summarized below. These and other embodiments are more fully described in the Detailed Description section. It is to be understood, however, that there is no intention to limit the invention to the forms described in this Summary of the Invention or in the Detailed Description. One skilled in the art can recognize that there are numerous modifications, equivalents and alternative constructions that fall within the spirit and scope of the invention as expressed in the claims.

One embodiment of the invention comprises a device for retracting internal tissue during an endoscopic surgical procedure. One device comprises a base section and an elongated section coupled to and extending away from the base section, the elongated section adapted to fit within a surgical portal and comprising an elongated section tip, a top section, an outer bottom shell, an inner cavity, and an extendable gripping mechanism. The extendable gripping mechanism is adapted to extend from the elongated section and then hold and release internal tissue. One extendable gripping mechanism comprises a biasing device and a telescoping section. The biasing device may be substantially located in the cavity and may have a proximal end operatively coupled to one of the base section and the outer shell. A distal end of the biasing device may be operatively coupled to the telescoping section.

Another embodiment of the present invention comprises method of retracting internal tissue. One method comprises creating a surgical portal, inserting a device into the surgical portal, extending a gripping mechanism from the device towards the internal tissue, capturing the internal tissue, at least partially retracting the gripping mechanism such that the internal tissue is removed from obstructing the surgical area, and setting the device in a position that allows for endoscopic instrument insertion into the surgical portal.

Yet another embodiment of the present invention comprises an internal tissue retraction system. One internal tissue reaction system comprises an internal tissue retraction device and a delivery device. One internal tissue retraction device comprises a base section and an elongated section coupled to and extending away from the base section, the elongated section comprising an extendable gripping mechanism adapted to hold and release internal tissue. One delivery device is removably coupled to the internal tissue retraction device and is adapted to set the internal tissue retraction device in place and operate the gripping mechanism.

These and other embodiments are described in further detail herein.

### BRIEF DESCRIPTION OF THE DRAWINGS

Various objects and advantages and a more complete understanding of the present invention are apparent and more readily appreciated by reference to the following Detailed Description and to the appended claims when taken in conjunction with the accompanying Drawings, wherein:

FIG. 1 is an isometric view of a device for retracting internal tissue during an endoscopic surgical procedure according to one embodiment of the invention;

FIG. 2 is a view of a right hip joint with a capsulotomy in the joint tissue;

FIG. 3 is a cross-sectional view of a device for retracting internal tissue during an endoscopic surgical procedure along line A-A of FIG. 1 according to one embodiment of the invention;

FIG. 4 is an isometric view of a device for retracting internal tissue during an endoscopic surgical procedure with an extended telescoping section and hook portion according to one embodiment of the invention;

FIG. 5 is an isometric view of a delivery device according to one embodiment of the invention;

FIG. 6 is an isometric view of an internal tissue retraction system according to one embodiment of the invention;

FIG. 7 is an isometric view of an internal tissue retraction system with an extended telescoping section and hook portion according to one embodiment of the invention;

FIG. 8A is an isometric view of an elongated section of a device for retracting internal tissue according to one embodiment of the invention;

FIG. 8B is an isometric front view of a transparent elongated section having an extended telescoping section of a device for retracting internal tissue according to one embodiment of the invention;

FIG. 9 is an end view of a base section without a cover of a device for retracting internal tissue according to one embodiment of the invention.

FIG. 10 is one embodiment of a method of retracting internal tissue.

### DETAILED DESCRIPTION

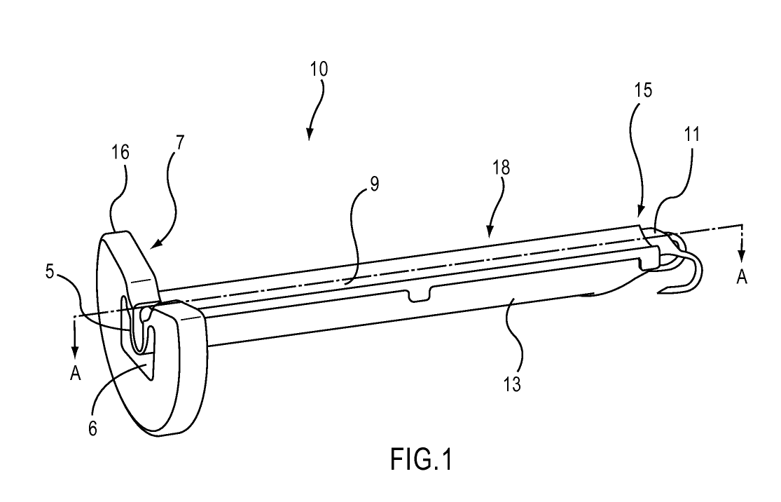

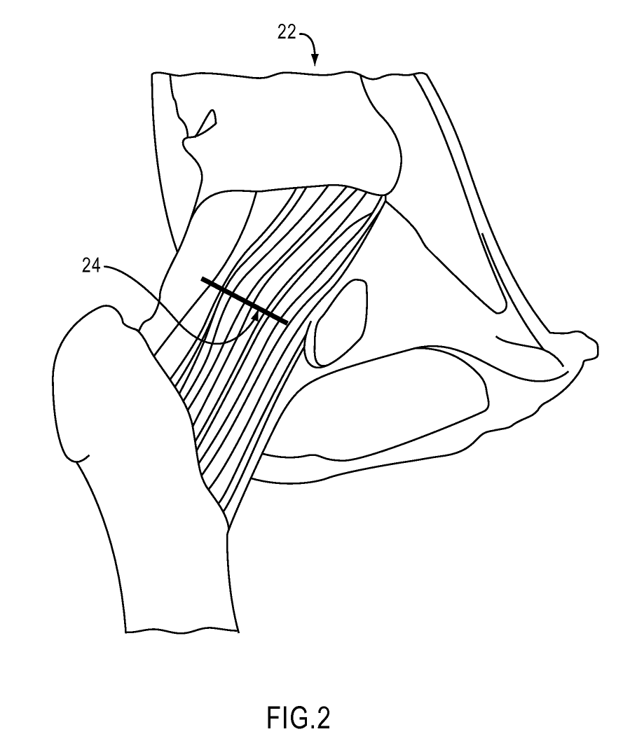

Referring now to the drawings, where like or similar elements are designated with identical reference numerals throughout the several views where appropriate, and referring in particular to FIG. 1, shown is a device 10 for retracting internal tissue during an endoscopic surgical procedure. The device 10 may be used during hip surgery such as hip arthroscopy procedures. As seen in FIG. 2, in one such surgical procedure, a capsulotomy 24 may be created in a right hip joint capsule 22. In creating the capsulotomy 24, or other surgical portal, the resultant free edges of the tissue—in this case, the hip joint capsule 22 tissue, may obstruct the field of view of an arthroscope and may impede the introduction and use of additional arthroscopic and endoscopic instruments. It is to be appreciated that the device 10 may also be used during laparascopy (for bowel retraction, for example) and other surgical procedures. It is to be appreciated that alternative shapes and sizes of the device in FIG. 1 are contemplated, such as a device having a slimmer elongated section 18. Such a device may minimize fluid leaks and minimize interference with additional instruments.

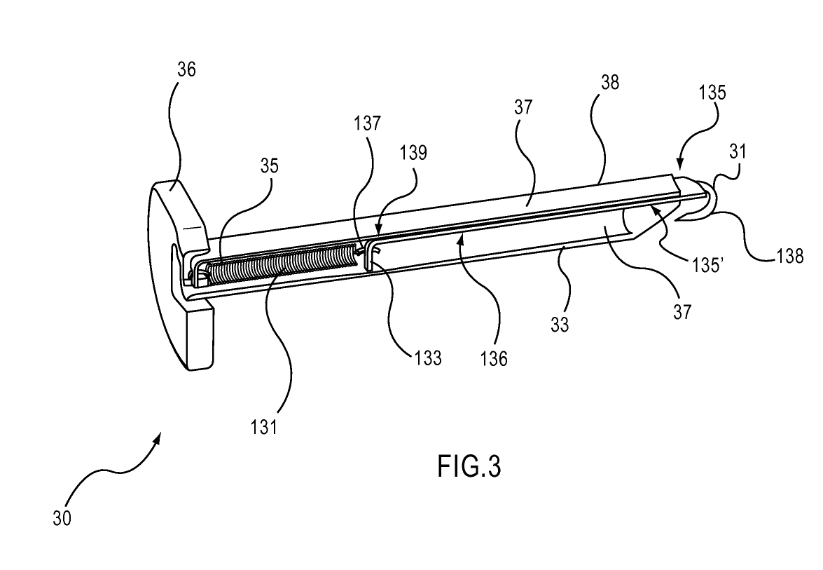

One embodiment of the device 10 is adapted to capture and retract internal tissue while not interfering with the use of other surgical instruments during the procedure. The device 10 is generally comprised of a base section 16 and the elongated section 18. The device 10 may be adapted to be securely left in place during the procedure upon retraction of the tissue for the duration of the procedure. In one embodiment, the elongated section 18 is adapted to be inserted and fit within the surgical portal. The elongated section 18 may be coupled or integrated to a center of the base section 16, substantially rigidly extending generally away from the base section 16 in a perpendicular manner. However, other non-perpendicularly-attached and non-rigidly coupled embodiments of elongated sections 18 are also contemplated. In FIG. 1, the elongated section 18 comprises an elongated section tip 15, a top section 9, an outer bottom shell 13 and an extendable gripping mechanism 11. The elongated section 38 may also comprise an inner cavity 37 as seen in FIG. 3.

With continuing reference to FIG. 3, the extendable gripping mechanism 31 is adapted to hold and release the internal tissue. One embodiment of the extendable gripping mechanism 31 may be comprised of a biasing device 131 and a telescoping section 133. The telescoping section may be comprised of an integrated shaft portion 136 and hook portion 31. The shaft portion 136 and hook portion 31 may also be coupled together. As seen in FIG. 3, the biasing device 131 may be substantially located in the cavity 37 and have a proximal end 35 operatively coupled to one of the base section 36, top section 9, and the outer shell 33. In FIG. 3, the biasing device 131 is coupled to a portion of the top side 39 that is bent downwardly towards the biasing device 131. The biasing device 131 may also have a distal end 137 operatively coupled to the telescoping section 133. One biasing device 131 may comprise a spring. However, the biasing device 131 may also comprise an elastomeric material or may comprise a suction vacuum pressure device such, but not limited to, a compressed air or compressed gas device. Additionally, the biasing device 131 may comprise a crank or a ratcheting mechanism adapted to create a state retraction force by varying the length of the elongated section 18 or an elongated section 18 component such as, but not limited to, the telescoping section 133. However, it is also appreciated that varying sizes of the different embodiments described herein are also contemplated. For example, multiple embodiment sizes may be required to accommodate a range of patients.

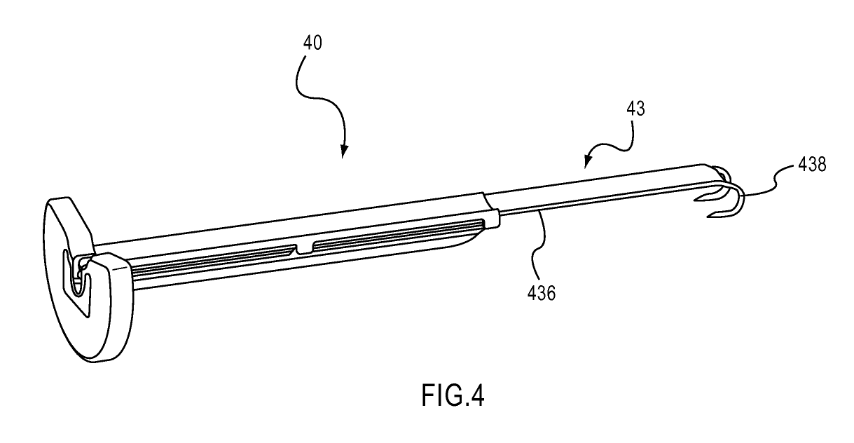

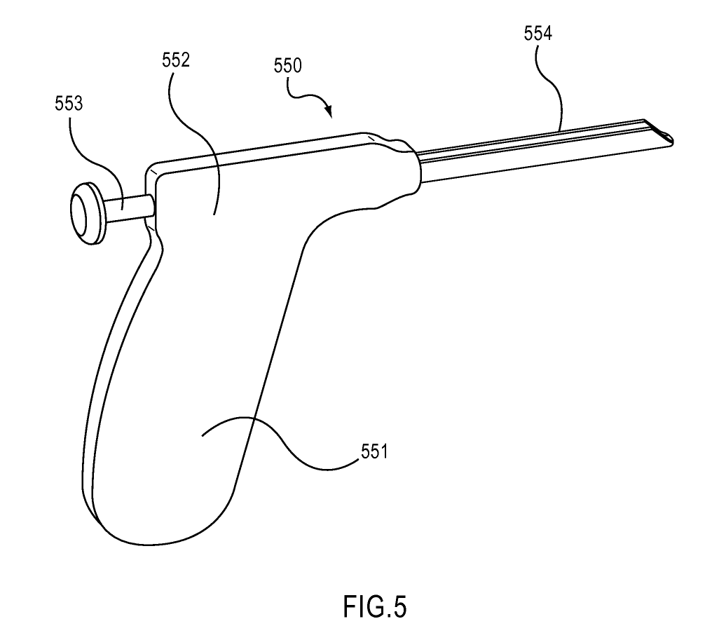

In one embodiment, the distal end 137 of the biasing device 131 is coupled to the shaft portion 136 of the telescoping section 133. One shaft portion 136 may be adapted to receive a longitudinally-aligned force. For example, a shaft portion proximal end 139 may be adapted to receive a force applied from a delivery device sufficient to overcome the retractive force applied to the telescoping portion 133 by the biasing device 131. In one such embodiment, the delivery device 550 of FIG. 5 may longitudinally extend a rod towards the shaft portion proximal end 139 and push the shaft portion proximal end 139 towards the elongated section tip 135. As the shaft portion proximal end 139 is extended towards the elongated section tip 135, the hook portion 138 may be positioned near the internal tissue. FIG. 4 shows one embodiment of a device 40 having an extended telescoping section 43 comprising a shaft portion 436 and hook portion 438. Returning to FIG. 3, upon release of the force from the telescoping section 133, the biasing device 131 retracts the telescoping section 133, allowing the hook portion 138 to capture the internal tissue and retract the tissue towards the elongated section 38.

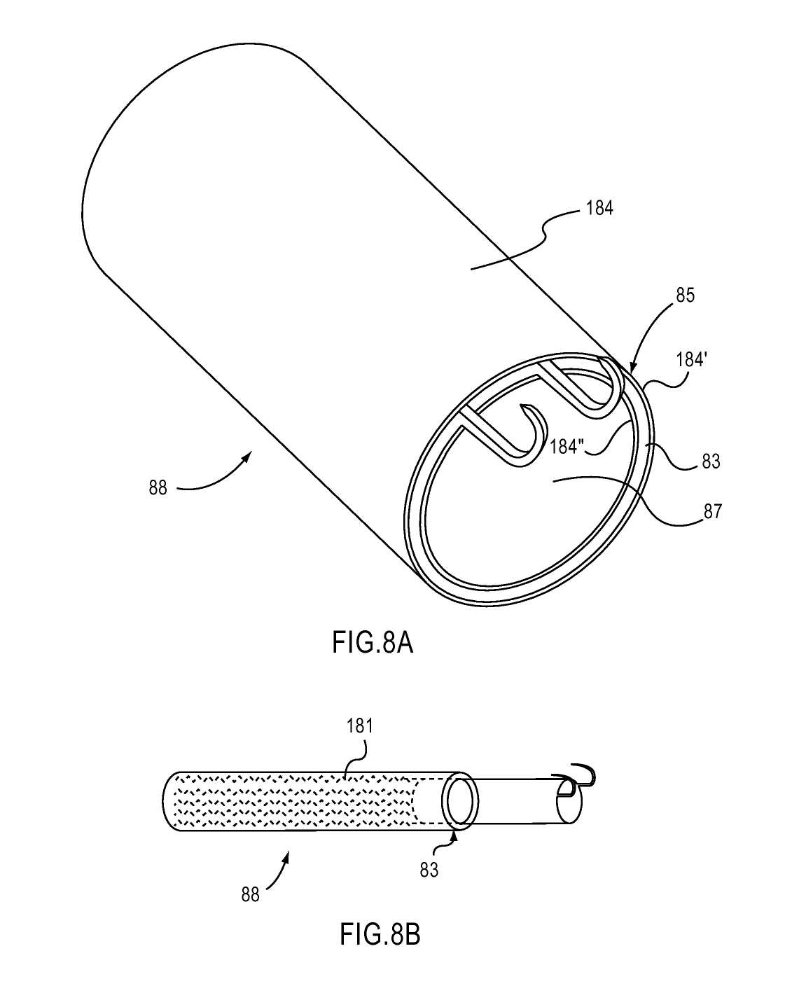

One top section 39 may be coupled to the outer bottom shell 133. The top section 39 may also be concave. The concavity of the top section 39 may allow the top section 39 to be used as a guide for the insertion of endoscopic instruments into the surgical area upon secure placement of the device 10. For example, instruments may be placed against the top section 39 and slid into position, with the concavity generally keeping the instruments in place. Other embodiments may comprise differently-shaped elongated sections that may be adapted for instrument insertion. For example, as seen in FIGS. 8A & 8B, the elongated section 88 may comprise an outer shell 184 having an outer side 184′ and an inner side 184″. Between the outer side 184′ and inner side 184″ may be an inner telescoping section 83 and a biasing device 181. The biasing device 181 may be comprised of multiple biasing devices 181 or it may comprise a circular biasing device in one embodiment. The inner cavity 87 may be adapted to receive surgical instruments. Similar to the embodiment seen in FIGS. 1 and 3, the telescoping section 83 is slidably extendable along a length of the elongated section 88 and adapted to extend past the elongated section tip 85.

Returning to FIGS. 1 and 3, in one embodiment, the base section 16 comprises a half-disc pad comprised of a polymeric foam. An inner surface 7 of the pad is adapted to contact a section of skin surrounding the surgical portal upon insertion of the elongated section 18 into the surgical portal. It is also contemplated that the pad could be replaced by adhesive strips which adhere to skin. Upon release of the force extending the telescoping section 133 towards the internal tissue, the retractive force of the biasing device 131 is applied to the telescoping section 133. This retractive force places the inner surface 7 against the skin, distributing the biasing device 131 retraction force to the skin. In this manner, the device 30 is securely held in place throughout the surgical procedure.

As seen in FIG. 3, the elongated section tip 135 may comprise an elongated section tip cavity exit 135′ through which the telescoping section 133 extends from the cavity 37 and past the elongated section tip 135, ending in the hook portion 138 in one embodiment. The elongated section tip cavity exit 135′ is bordered by the top section 39 and the outer bottom shell 33. As the force is applied to and released from the telescoping section 133, and the hook portion 138 extends away from and subsequently retracts towards the elongated section tip 135, the telescoping section 133 slidably operates through this elongated section tip cavity exit 135′.

Although the gripping mechanism 31 in FIG. 3 and elsewhere shows the use of one or more metal tissue hooks as a hook portion 138, it is to be appreciated that other items besides tissue hooks such as, but not limited to a clasping device, paddles, balloon structures, etc. may also be used in connection with the gripping mechanism 31. Additionally, embodiments having a metal top section 39 and outer bottom shell 33 are contemplated, as are a polymeric top side 39 and outer bottom shell 33, or any other suitable metal, polymeric, or elastomeric material.

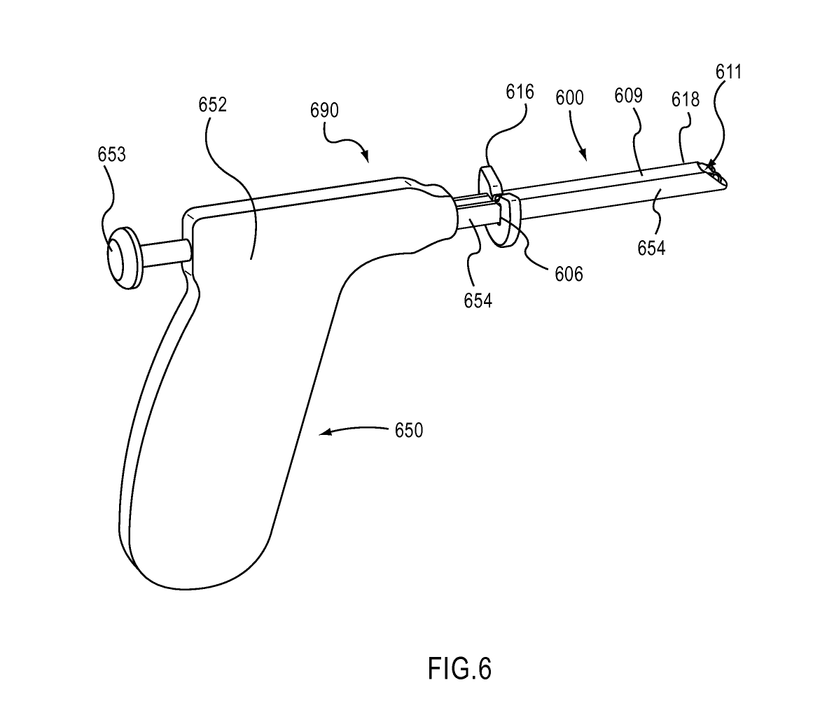

As seen in FIG. 6, another embodiment of the invention comprises an internal tissue retraction system 690. One embodiment of the internal tissue retraction system 690 comprises an internal tissue retraction device 600 and a delivery device 650. The internal tissue retraction device 600 may comprise the device as seen in FIGS. 1, 3, and 4. For example, the internal tissue retraction device 600 may comprise a base section 616 and an elongated section 618 coupled to and extending away from the base section 616, the elongated section 618 comprising a gripping mechanism 611 adapted to hold and release internal tissue. The delivery device 650 is adapted to removably couple to the internal tissue retraction device 600 and operate the gripping mechanism 611.

As seen in FIG. 5, one delivery device 550 comprises a handle 551, a housing 552 integrated to the handle 551, an actuator 553 coupled to the housing 552, and a receptor extension 554 coupled to the housing and adapted to receive the internal tissue retraction device 600. As seen in FIG. 1, the base section 16 of one embodiment of an internal tissue retraction device 10 comprises a bore 6. The bore 6 may be U-shaped. One of the sides of the U-shaped bore 6 (or any other bore shape) may be coupled or integrated to the elongated section 18. For example, the inner side 5 of the bore 6 may be coupled or integrated into the outer bottom shell 13 of the elongated section 18.

The U-shape bore 6 of the base section 16 and the outer bottom shell 13 are adapted to receive the receptor extension 554. As seen in FIG. 6, in one embodiment of an internal tissue retraction system 690, the receptor extension 654 fits through the base section bore 606. The receptor extension 654 then fits around the outer bottom shell (seen as element number 13 in FIG. 1—not shown in FIGS. 5 & 6). Therefore, upon insertion of the extension 654 into the bore 606, the internal tissue retraction device 600 rests within the receptor extension 654. The internal tissue retraction device 600 may be removably locked into place on the receptor extension 654. For example, in one embodiment, a locking mechanism may couple the internal tissue retraction device 600 onto the receptor extension 654. Upon operation of the actuator 653 or another trigger on the delivery device 650, the internal tissue retraction device 600 may be unlocked from the delivery device 650 such that the delivery device 650 may be removed from the internal tissue retraction device 600 by sliding the receptor extension 654 back through the bore 606.

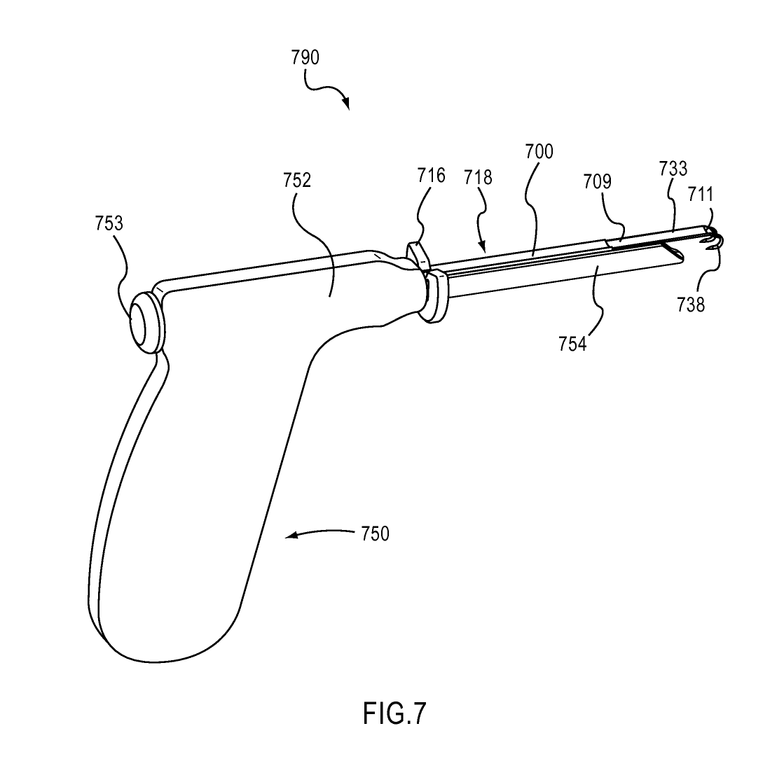

FIG. 7 shows one final loading position of the internal tissue retraction device 600 on the delivery device 650. In FIG. 7, base section 716 is located proximal the housing 752. Upon loading the internal tissue retraction device 700 onto the delivery device 750, the receptor extension 754 and loaded elongated section 718 are inserted into the surgical portal. The actuator is engaged and the gripping mechanism 711 is placed near the internal tissue. For example, the actuator 753 in FIG. 7 is depressed towards the housing 752, extending the telescoping section 733 and gripping mechanism 711. In one embodiment, the actuator 753 may comprise a button operatively coupled to a shaft extending through the housing 753 to the receptor extension 754. Once the gripping mechanism 711 is placed proximal the internal tissue, the actuator 753 is released. Upon release of the actuator 753, in one embodiment, the hook portion 738 is retracted towards the base section 716 by the biasing device 131 (as seen in FIG. 3), gripping and retracting the internal tissue. Upon proper retraction of the internal tissue, the internal tissue retraction device 700 is set in place and the delivery device 750 is de-coupled from the internal tissue retraction device 700. Upon removal of the delivery device 750, the concave top section 709 and telescoping section 733 may be used as a guide for passing instruments into the surgical area.

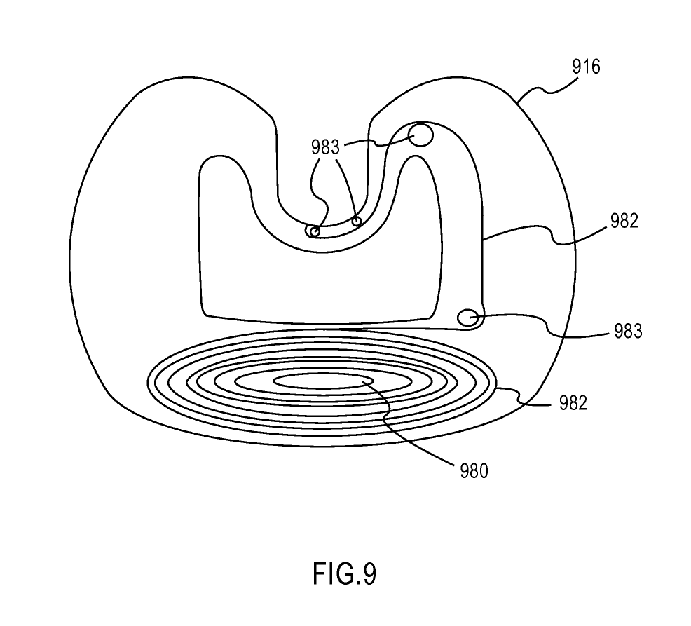

As seen in FIG. 9, one embodiment may comprise a base section 916 having a spool 980. The spool 980 may comprise a torsion spring. A flexible cable 982 may extend from the spool 980 to one or more rollers 983. The flexible cable may be wound around the spool 980 and may exit the base section 916 to the telescoping section 133 (as seen in FIG. 3, not shown in FIG. 9). The FIG. 9 base section 916 may replace the biasing device 131 of FIG. 3. Similar to the device 700 seen in FIG. 7 and elsewhere throughout the specification, after operation and release of the actuator 753, the torsion spring may retract the cable 982 and telescoping section 133 towards the base section 916, allowing the gripping mechanism 711 to retract the internal tissue. A device 700 having a spool 980 similar to the spool 980 seen in FIG. 9 may allow for a slimmer profile of the elongated section 718 and receptor extension 754, thereby allowing for easier introduction of instruments into the surgical portal and diminishing the obstruction of the surgical area by the elongated section 718. The cable 982 may also comprise a wire, monofilament, etc.

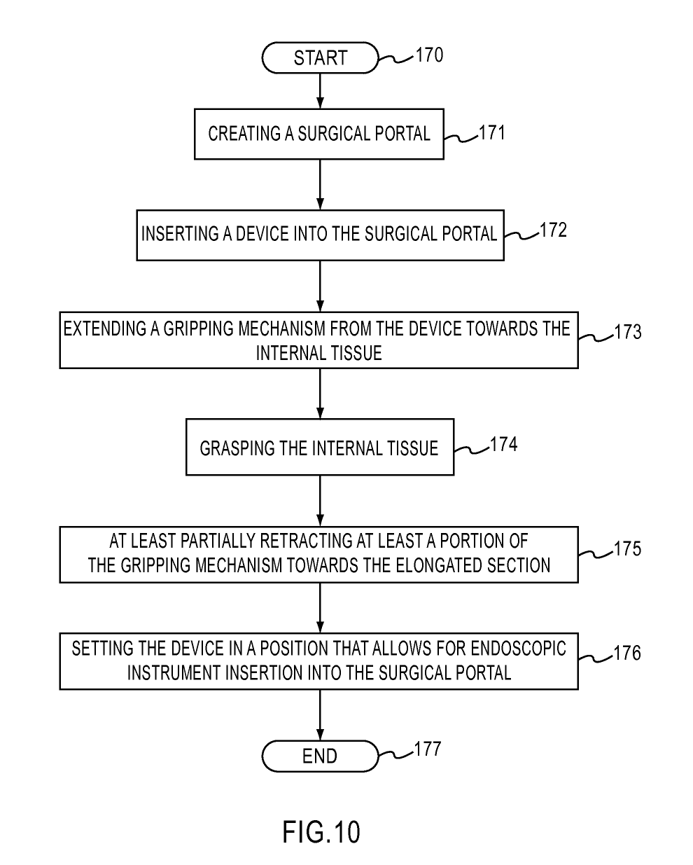

FIG. 10 depicts a method of refracting internal tissue using a device constructed in accordance with one or aspects disclosed herein. The method begins at 170 and at 171 comprises creating a surgical portal. For example, a surgical portal such as, but not limited to, a capsulotomy similar to the capsulotomy 24 seen in FIG. 2 may be created, among other surgical portals. Anterolateral and mid-anterior portals into a joint capsule may also be created. Upon creating the surgical portal, at 172, the method comprises inserting a device into the surgical portal. One device that may be inserted into a surgical portal such as the capsulotomy 24 comprises the internal tissue refraction device 10 seen in FIG. 1. For example, the elongated section 18 of the internal tissue retraction device 10 may be placed into the capsulotomy 24 with the help of the delivery device 550 of FIG. 5. Other devices are also contemplated.

At 173, the method comprises extending a gripping mechanism from the device towards the internal tissue. This may comprise using the delivery device 550 to extend the gripping mechanism 711 from the device 700, as seen in the FIG. 7 and described above. Subsequently, at 174, the method comprises grasping the internal tissue. For example, grasping the internal tissue may comprise retracting the grasping mechanism 711 to pierce the internal tissue with an integrated hook portion 738. However, grasping the internal tissue may also relate to the use of a clamping device, a suction device, or any other device type that is known in the art which may be used to capture internal tissue.

At 175, the method comprises at least partially retracting at least a portion of the gripping mechanism towards the elongated section. For example, as previously explained and as seen in FIG. 3, one gripping mechanism 31 may comprise a biasing device 131 and a telescoping section 133. Upon application of a longitudinally-aligned force on the telescoping section 133, the telescoping section 133 slidably moves through the elongated section tip cavity exit 135′ as the integrated hook portion 138 moves away from the elongated section tip 135. The biasing device 131 extends and imparts an opposing retracting force on the telescoping section 133. At least partially retracting at least a portion of the gripping mechanism 31 may refer to only partially retracting the telescoping section 133 and integrated hook portion 138 since at least one biasing device type (such as, a pressure-inducing biasing device) may not need to be retracted.

In one example, when the hook portion 138 captures the internal tissue, a portion of the internal tissue may continue to be coupled to the joint. Therefore, the tissue applies an opposing force to the biasing device 131, thereby allowing the telescoping section 133 to be partially extended. Upon release of the internal tissue, the entire gripping mechanism 31 may be fully retracted to an initial position.

At 176, the method comprises setting the device in a position that allows for endoscopic instrument insertion into the surgical portal. In this step, upon retraction of the internal tissue, a delivery device such as, but not limited to, the delivery device 550 seen in FIG. 5 may place the internal tissue retraction device 10 in secure position where the base section 16 rests against the skin surrounding the surgical portal. Upon ensuring that the internal tissue is properly retracted and the device 10 is securely in place, the delivery device 550 is de-coupled from the device 10. One method of determining whether the internal tissue is properly retracted prior to de-coupling the delivery device 550, is to move the internal tissue retraction device 10 and determine whether the tissue will be removed from the gripping mechanism 11 with additional movement. As the internal tissue retraction device 10 often moves during the surgical procedure, it is important to ensure that the tissue will not become dislodged from the gripping mechanism 11 for the range of movement that the internal tissue retraction device 10 will encounter during surgery. One method may move the internal tissue retraction device 10 in a longitudinal direction to ensure that the biasing device 131 is properly operating so that when the device 10 is moved during the surgical procedure, the tissue does not become dislodged from the gripping mechanism 11. Upon determining that the device 10 is securely in place, instruments may be inserted into the surgical portal and the surgical procedure may be completed as per the standard of care (capsule closed, etc.). Surgical instruments may then be slid along a top side 39 of the elongated section to the surgical area. However, it is also contemplated that instruments may be inserted into the portal with the delivery device 550 attached.

As seen in FIGS. 8A & 8B, the elongated section 88 may comprise an outer shell 184 having an outer side 184′ and an inner side 184″. Between the outer side 184′ and inner side 184″ may be an inner telescoping section 83 and a biasing device 181. Together, the outer shell 184 and inner telescoping section 83 may be used as a cannula to pass instruments to the surgical area. Likewise, the delivery device 550 of FIG. 5 may also comprise a cannula adapted to pass instruments to the surgical area.

In order to properly remove the device from the surgical area, the delivery device 550 may be re-coupled to the device 10. Thereupon, the internal tissue may be released from the device by operating the delivery device 550. For example, the delivery device 500 may extend the gripping mechanism so that the internal tissue slides off of the gripping mechanism 11. Other methods are contemplated. Upon releasing the tissue from the internal tissue retraction device 10, both the internal tissue retraction device 10 and the delivery device 550 may be removed from the portal.

When the delivery device 750 is used to extend the gripping mechanism 711 from the elongated section 718 towards the internal tissue and at least partially retract the gripping mechanism 711, in one method, pressure is applied to the actuator 753, as seen in FIG. 7. The actuator 753 may be coupled to a rod located internally to the housing 752 of the delivery device 750. The rod may also be coupled to the gripping mechanism 711. Therefore, as pressure is applied to the actuator 753 and the actuator 753 is moved from a first position to a second position, the gripping mechanism is engaged by the actuator. One first position of the actuator 653 is seen in FIG. 6 and one second position of the actuator 753 is seen in FIG. 7. Upon releasing the pressure from the actuator, the gripping mechanism engages with the internal tissue, retracting the internal tissue and creating a larger field of view for an endoscope.

Those skilled in the art can readily recognize that numerous variations and substitutions may be made in the invention, its use and its configuration to achieve substantially the same results as achieved by the embodiments described herein. Accordingly, there is no intention to limit the invention to the disclosed exemplary forms. Many variations, modifications and alternative constructions fall within the scope and spirit of the disclosed invention.

What is claimed is:

1. A device for retracting internal tissue during an endoscopic surgical procedure comprising,
a base section; and
an elongated section adapted to fit within a surgical portal, the elongated section coupled to and extending from the base section, the elongated section comprising,
an elongated section tip,
a top section;
an outer bottom shell,
an inner cavity, and
an extendable gripping mechanism adapted to hold and release internal tissue, the extendable gripping mechanism comprising,
a telescoping section, and
a biasing device substantially located in the cavity, the biasing device comprising,
a proximal end operatively coupled to one of the base section, top section, and the outer shell, and
a distal end operatively coupled to the telescoping section.

2. The device of claim 1 wherein,
the elongated section tip comprises an elongated section tip cavity exit;
the telescoping section is slidably extendable along a length of the elongated section through the elongated section tip cavity exit;
the biasing device is adapted to operatively impart a force on the base section; and
the base section comprises a pad adapted to,
contact a section of skin surrounding the surgical portal, and
distribute the force to the section of skin.

3. The device of claim 1 wherein,
the extendable gripping mechanism further comprises one of more tissue hooks, and wherein the biasing device comprises at least one of an elastomeric material and a spring.

4. The device of claim 1 wherein the surgical portal comprises one of a hip tissue capsulotomy, an arthroscopic, and a laparoscopic portal.

5. The device of claim 1 wherein,
the elongated section is generally coupled to a center of a half-disc shaped base section;
and the gripping mechanism is adapted to capture a free edge of the tissue.

6. A method of retracting internal tissue comprising,
creating a surgical portal;
inserting a device into the surgical portal;
extending a gripping mechanism from the device towards the internal tissue;
grasping the internal tissue;
at least partially retracting at least a portion of the gripping mechanism and internal tissue towards the elongated section; and
setting the device in a position that allows for endoscopic instrument insertion into the surgical portal

7. The method of claim 6 wherein,
inserting a device into the surgical portal comprises inserting an elongated section of an internal tissue retraction device into the surgical portal; and further comprising,
coupling a delivery device to the internal tissue retraction device prior to inserting the elongated section of the internal tissue retraction device into the surgical portal, and
using the delivery device to,
extend the gripping mechanism from the elongated section towards the internal tissue, and
at least partially retract a portion of the gripping mechanism towards the elongated section.

8. The method of claim 7, further comprising,
decoupling the delivery device from the internal tissue retraction device; and
completing the surgical procedure.

9. The method of claim 8 further comprising,
recoupling the delivery device to the internal tissue retraction device; and
repositioning the internal tissue retraction device.

10. The method of claim 7 further comprising, using the delivery device to release the tissue from the gripping mechanism.

11. The method of claim 7 wherein,
at least one of the delivery device and the internal tissue retraction device comprises a cannula; and further comprising, passing instruments through the cannula.

12. The method of claim 6, further comprising, using the elongated section as a guide for passing instruments to a surgical area.

13. The method of claim 8 further comprising,
longitudinally moving at least a portion of the tissue retraction device; and
retaining the grasp of the internal tissue.

14. The method of claim 7 wherein using the delivery device to extend the gripping mechanism from the elongated section towards the internal tissue and at least partially retract a portion of the gripping mechanism towards the elongated section comprises,
applying pressure to an actuator;
moving the actuator from a first position to a second position;
releasing the pressure from the actuator; and
using the gripping mechanism to retract the internal tissue.

15. The method of claim 6 wherein, at least partially retracting at least a portion of the gripping mechanism and internal tissue towards the elongated section comprises creating a larger field of view for an endoscope.

16. The method of claim 6 wherein, the surgical portal comprises at least one of an anterolateral and mid-anterior portal into a joint capsule and a capsulotomy.

17. An internal tissue retraction system comprising,
a tissue retraction device, the tissue retraction device comprising,
a base section, and
an elongated section coupled to and extending from the base section, the elongated section comprising a gripping mechanism adapted to hold, retract, and release internal tissue; and
a delivery device removably coupled to the tissue retraction device and adapted to operate the gripping mechanism.

18. The system of claim 17 wherein the internal tissue retraction system further comprises, a biasing device housed proximal the base section, the biasing device operatively coupled to the gripping mechanism.

19. The system of claim 18 wherein, the biasing device comprises a torsion spring substantially enclosed by the base section.

20. The system of claim 17 wherein, the elongated section comprises a top section, an outer bottom shell, a telescoping section, and a biasing device coupled to the telescoping section.

21. The system of claim 17 wherein, the delivery device comprises,
a handle;
a housing integrated to the handle;
an actuator coupled to the housing; and
a receptor extension coupled to the housing and adapted to receive the internal tissue retraction device.

22. The system of claim 21 wherein, the actuator comprises a button operatively coupled to a shaft extending through the housing to the receptor extension.
